(19) United States
(12) Patent Application Publication
(10) Pub. No.: US 2021/0022678 A1
(43) Pub. Date: Jan. 28, 2021

(54) WEARABLE HEALTH MONITORING DEVICES AND METHODS

(71) Applicant: Health Alert, LLC, Angwin, CA (US)

(72) Inventors: Alois Antonin Langer, Harrison City, PA (US); Walter Louis Seale, Angwin, CA (US)

(21) Appl. No.: 16/936,317

(22) Filed: Jul. 22, 2020

Related U.S. Application Data
(60) Provisional application No. 62/877,657, filed on Jul. 23, 2019.

(51) Int. Cl. A61B 5/00; A61B 5/024; A61B 5/0255

(52) U.S. Cl. CPC A61B 5/6843; A61B 5/681; A61B 5/0255; A61B 5/02416; A61B 5/02438; A61B 5/6898

(57) ABSTRACT

Personal health monitoring devices and methods are disclosed. A health monitoring device includes a sensor unit including a source and receiver of optical energy, a housing configured to receive and house the sensor unit, and a coupling mechanism configured to couple the housing to a band to be worn at least partially about one of a wrist or a lower forearm of a wearer of the band. The coupling mechanism is further configured to position the housing pointing the sensor unit inward to the wrist or lower forearm of the wearer with an inward radial force on the sensor unit based on tension in the band. The health monitoring device also includes a data storage device and a processor configured to receive, from the sensor unit, data about received optical energy, and to produce photoplethysmogram data using the received data, and to store data to the data storage device.

### CROSS-REFERENCE TO RELATED APPLICATION

This application claims priority to U.S. Provisional Application No. 62/877,657, filed on Jul. 23, 2019, and titled “Wearable Health Monitoring Devices and Methods,” which is hereby incorporated by reference in its entirety.

### TECHNICAL FIELD

The present disclosure relates generally to the field of personal health systems. More specifically, the present disclosure relates to personal health monitoring using wearable health monitoring devices and methods.

### BRIEF DESCRIPTION OF THE DRAWINGS

The present embodiments will become more fully apparent from the following description and appended claims, taken in conjunction with the accompanying drawings. Understanding that the accompanying drawings depict only typical embodiments and are, therefore, not to be considered limiting of the scope of the disclosure, the embodiments will be described and explained with specificity and detail in reference to the accompanying drawings.

FIG. 1 is a partial schematic diagram of a wearable health monitoring device, according to one embodiment, including a circuit with a light emitting diode (LED).

FIG. 2 is a partial schematic diagram of a detection circuit of a wearable health monitoring device, according to an embodiment of the present disclosure, such as the wearable health monitoring device of FIG. 1.

FIG. 3A is an illustration of a sample portion of a photoplethysmogram such as may be produced from interpretive data of the microcontroller of FIG. 2.

FIG. 3B is an illustration of a sample portion of a photoplethysmogram such as may be produced from interpretive data of the microcontroller of FIG. 2 and indicative of anomalous cardiac rhythm.

FIG. 3C is an illustration of a sample portion of a photoplethysmogram such as may be produced from interpretive data of the microcontroller of FIG. 2 and comprising a waveform having a probable movement artifact.

FIG. 4A is a side view of the wearable personal health monitoring device of FIGS. 1 and 2, according to an embodiment that is improved upon by the present disclosure.

FIG. 4B is a side view of the wearable personal health monitoring device of FIGS. 1 and 2, according to an embodiment of the present disclosure, and that is positioned relative to a band to allow an inward radial force produced by the band to press the wearable personal health monitoring device toward the skin of the wearer.

FIG. 5A is a partial side view of a wearable health monitoring device having loop connectors to dispose and retain a housing and a sensor unit against the skin of the wearer, according to an embodiment of the present disclosure.

FIG. 5B is a partial bottom view of the wearable health monitoring device of FIG. 5A, according to an embodiment of the present disclosure.

FIG. 5C is a partial bottom view of the wearable health monitoring device of FIG. 5A having loop connectors, according to an embodiment of the present disclosure.

FIG. 6 is a partial side view of a wearable health monitoring device according to an embodiment of the disclosure.

FIG. 7A is a partial inverse plan view of a wearable health monitoring device, according to an embodiment of the disclosure.

FIG. 7B is a partial side view of the wearable health monitoring device of FIG. 7A, according to an embodiment of the present disclosure.

FIG. 8A is a perspective view of a wearable health monitoring device, according to an embodiment of the present disclosure, and with a housing having an arcuate form.

FIG. 8B is a partial side view of the wearable health monitoring device of FIG. 8A.

FIG. 9 is a schematic of a method of a wearable health monitoring device that, in at least some ways, may be used with each of the embodiments disclosed herein.

### DETAILED DESCRIPTION

Individuals may realize substantial health and medical care benefits through long-term electronic monitoring, e.g. of disease processes related to medical conditions. One example where such benefits can be realized is through continuous transcutaneous blood sugar monitoring for a diabetic individual. Continuous transcutaneous blood sugar monitoring may be achievable via a portable device that an individual carries or wears. Similarly, continuous heart monitoring may benefit an individual having a heart disease, or to a person prone to various life-threatening conditions, e.g., arrhythmias, etc. A health monitoring device, such as a wearable health monitoring device, affording minimal impact on an individual's daily routine while offering continuous monitoring may be highly effective in collecting health-related data. Furthermore, while such a non-intrusive wearable device may be oriented toward collecting medical information for an identified medical issue, the device may simultaneously perform continuous health data monitoring and collection. A wearable device that provides a high degree of continuous monitoring through reliable positioning against the skin of a wearer is of particular utility. A non-intrusive wearable health monitoring device may further be enabled to provide timely notification to a wearer and/or another person (e.g., household member, family member, bystander, caregiver, medical care provider, etc.), including a layperson or an otherwise unaware person, of an anomalous event for which immediate attention may be warranted and may be further configured to provide ongoing monitoring of the anomalous event. Such a device may further be coupled with a system for administration of medicine, therapy, or other appropriate protocol upon detection of a trigger event.

It will be readily understood that the components of the embodiments as generally described and illustrated in the figures herein could be arranged and designed in a wide variety of different configurations. Thus, the following more detailed description of various embodiments, as represented in the figures, is not intended to limit the scope of the disclosure, as claimed, but is merely representative of various embodiments. While the various aspects of the embodiments are presented in drawings, the drawings are not necessarily drawn to scale unless specifically indicated.

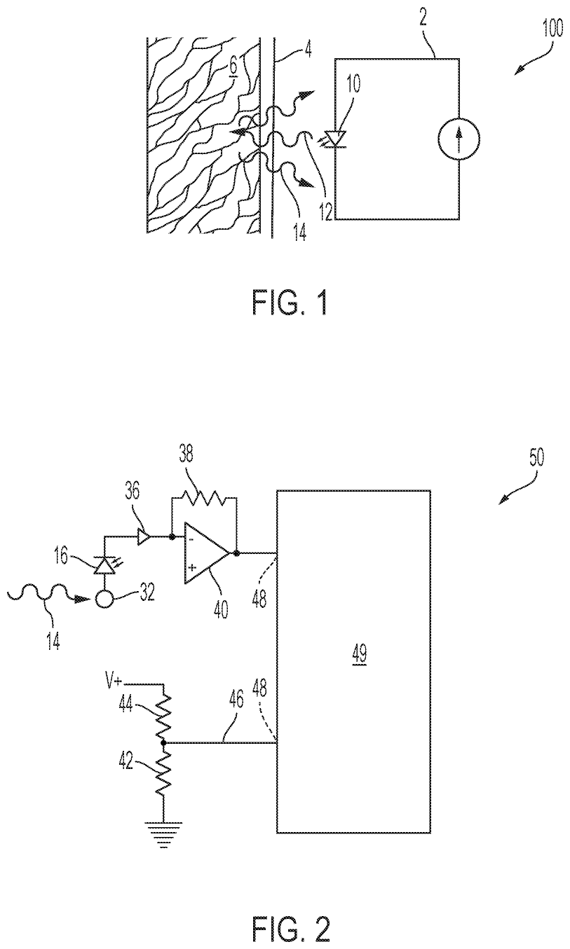

FIG. 1 is a schematic diagram of a wearable health monitoring device 100 having a circuit 2 with a light emitting diode (LED) 10. The circuit 2 is near a portion of skin 4 of a wearer, with the LED 10 oriented toward the skin 4. The skin 4 overlies a region of subdermal matter 6. With the circuit 2 is powered, the LED 10 may radiate light 12 toward the skin 4. The light 12 may be of a suitable wavelength and amplitude for a diagnostic purpose. The light 12 may at least partially penetrate the skin 4 to reach the underlying subdermal matter 6. Throughout this disclosure, representations in various figures of the skin 4 and subdermal matter 6 are not to scale, nor necessarily representative of a particular feature, but are provided for conceptual reference. The subdermal matter 6 may comprise vasculature, musculature, etc. The subdermal matter 6 may diffuse and reflect a portion of the light (diffused/reflected light) 14. The diffused/reflected light 14 may also be attenuated or altered in wavelength and/or amplitude by the subdermal matter 6. Changes within the subdermal matter 6 may produce varied attenuation or alterations in the wavelength and/or amplitude of the diffused/reflected light 14. More particularly, variations within the vasculature of the subdermal matter 6 may produce identifiable variations in wavelength and/or amplitude of the diffused/reflected light 14. For example, vascular blood pressure varies from moment to moment as the heart pumps blood into the gross vasculature. Differences in vascular blood pressure may identifiably alter the diffused/reflected light 14.

FIG. 2 is schematic diagram of a detection circuit 50, according to an embodiment of the present disclosure, such as may be used with the wearable health monitoring device 100 of FIG. 1. The detection circuit 50 comprises a processor 49 such as a microcontroller, a programmable logic controller (PLC), a field programmable gate array (FPGA), a central processing unit (CPU), other processors, or combinations thereof. Reflected diffused light 14 may instigate a current 36 through a photodiode 16 such that the photo-electric current 36 is delivered to an inverting input of the transconductance amplifier 40. Although not shown, the non-inverting input of the transconductance amplifier 40 may be connected to a DC voltage, such as ground (zero Volts). A first resistor 38 is coupled between the inverting input of the transconductance amplifier 40 and an output of the transconductance amplifier 40, which is connected to the processor 49 at an analog to digital (A-D) input channel 48. The transconductance amplifier 40 the current 36 into a voltage for the A-D input channel 48. In the present disclosure, the photo-electric current 36 may result from the diffused/reflected light (see 14 in FIG. 1) striking the photodiode 16. More particularly, variations in the diffused/reflected light 14 may cause variations (e.g., proportional variations) in the photo-electric current 36 produced at the photodiode 16. In one embodiment, voltage may also be passed through a resistor 44, which couples to a pressure-sensitive resistor 42 acting as a voltage divider such that voltage 46 is directed to another A-D input channel 48 of the processor 49 based on (e.g., proportional to) the pressure applied to the pressure-sensitive resistor 42. Pressure may be applied to the pressure-sensitive resistor 42 by securing an associated wearable health monitoring device to a wearer and the voltage 46. Vp will be proportional to the pressure.

The processor 49 may comprise a processing element and a data storage device (such as a memory), or a processing element coupled to an external data storage device/memory containing computer-readable instructions. The processor 49 may be configured to access, receive, and execute the computer-readable instructions to record, to a memory, interpretive data of the signal received from the photodiode 16 and the signal received from the pressure-sensitive resistor 42. The interpretive data may, at the processor 49 or at an external computing device, be rendered for presentation via an output device in a meaningful way to the wearer, a caregiver, a medical care provider, etc.

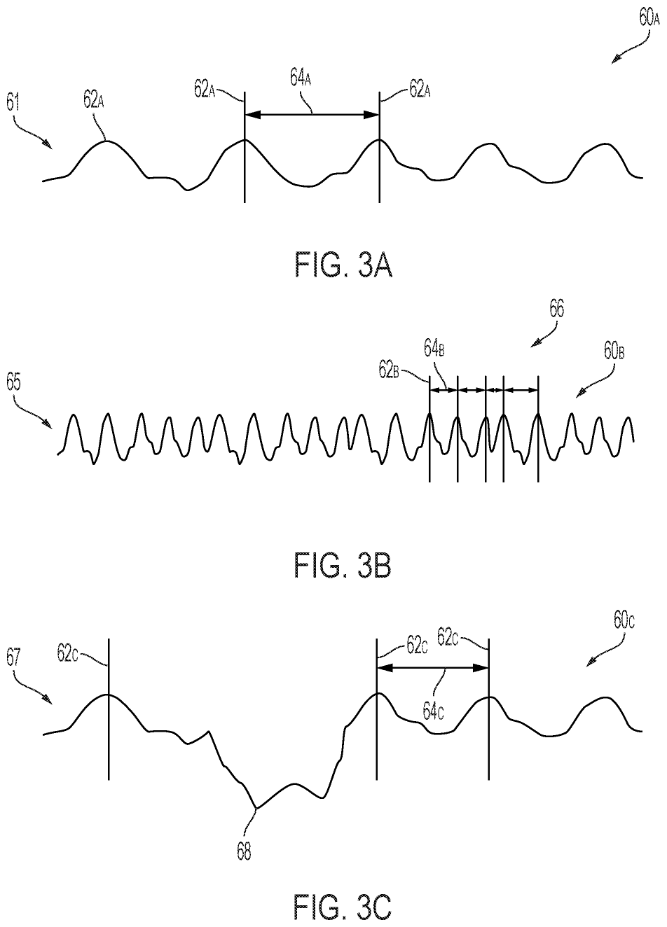

FIG. 3A is an illustration of a sample portion of a photoplethysmogram 60A such as may be produced from interpretive data of the processor 49 of FIG. 2. The sample portion of the photoplethysmogram 60A comprises a waveform 61. The waveform 61 includes peaks 62A each representing momentary maximal signal strength from the photodiode (see 16 in FIG. 2) resulting from a vascular change of the subdermal matter (see 6 in FIG. 1) caused by, nominally, a left ventricular compression of the heart of a wearer of a wearable personal health monitoring device. A period (duration between two consecutive peaks), such as the period 64A, may be measurable and diagnostic. Similarly, a frequency, i.e., number of peaks 62A in a selected duration or time, may be similarly diagnostic. An amplitude of a peak 62A may also be diagnostic. The sample portion of the photoplethysmogram 60A may represent a nominal cardiac rhythm for a duration of approximately five seconds.

FIG. 3B is an illustration of a sample portion of a photoplethysmogram 60B such as may be produced from interpretive data of the processor 49 of FIG. 2 and indicative of anomalous cardiac rhythm. The sample portion of the photoplethysmogram 60B may be a continuation of the sample portion of the photoplethysmogram 60A of FIG. 3A. The sample portion of the photoplethysmogram 60B comprises a waveform 65 that includes peaks 62B, and may represent a duration of approximately five seconds. As will be noted by inspecting FIG. 3B, the waveform 65 represents a very high heartrate on the order of about 230 beats per minute. A period of time 66 approximately equal to the peak-to-peak period 64A of FIG. 3A is shown, and within which a number of peaks 62B occur having much shorter periods 64B than the period 64A of FIG. 3A. The shorter periods 64B of the sample portion of the photoplethysmogram 60B of FIG. 3B may be anomalous and diagnostic. While the sample portion of the photoplethysmogram 60B may be diagnostic of particular vascular and cardiac issues, variations in the waveform may be diagnostic of other health issues and useful for, by way of example, measuring oxygen saturation, blood pressure, autonomic functions, blood chemistry changes, etc.

FIG. 3C is a sample portion of a photoplethysmogram 60C comprising a waveform 67 having a probable movement artifact 68. The sample portion of the photoplethysmogram 60C may be a continuation of the sample portion of the photoplethysmogram 60A of FIG. 3A or the photoplethysmogram 60B of FIG. 3B recorded at a different time, and represents a duration of approximately five seconds. The sample portion of the photoplethysmogram 60C comprises a waveform 67 having peaks 62C with a similar amplitude and frequency as those of the sample portion of the photoplethysmogram 60A of FIG. 3A. The waveform 67 also contains an anomalous portion between a first peak 62C of a period 64C and the peak 62C prior to the first peak 62C of the period 64C. The anomalous portion of the waveform 67 may be a probable movement artifact 68 produced by a relatively radical drop in signal strength from the photodiode (see 16 in FIG. 2). The relatively radical drop in signal strength from the photodiode 16 may be caused by the wearable health monitoring device momentarily moving somewhat away from the skin (see 4 in FIG. 1) of the wearer, possibly as a result of strenuous physical movement, e.g., exercise, a fall, etc. A capability to record probable movement artifacts may aid diagnostically by enabling omission of non-diagnostic data, or may indirectly suggest an activity or event that may have triggered or been related to diagnostically significant data. In other words, the processor may evaluate the photoplethysmogram data to assess a health status of the wearer.

The processer may be configured to receive data from the sensor about received optical energy, and to produce photoplethysmogram data using the received optical energy data. The processor may store the photoplethysmogram data, the health status, or both at the data storage device/memory, or to an external device, and may do so in real-time or near real-time. In some embodiments the processor may transfer the photoplethysmogram data, the health status, or both to an external device at a later time (e.g., when electrical communication between the processor and the external device is established). The external device may be a “smartwatch” worn by the wearer of the wearable personal health monitoring device, a “smart phone” or cellular telephone carried by the wearer, a tablet computer, a laptop or desktop computer, or a similar device. The external device may be capable of communicating with the wearable personal health monitoring device in order to receive and store data of the sensor unit, or data stored at the data storage device/memory.

The processor may be configured to evaluate photoplethysmogram data to assess a health status of the wearer, for example, a variety of health conditions, such as, for example, tachycardia, atrial fibrillation, etc. Because the wearable health monitoring device can be worn continuously for hours or days at a time, and for weeks or months with only brief interruption, a capacity to detect transient conditions, in particular, may be greatly enhanced. An ability to readily adjust the fit of the wearable health monitoring device, as described herein, may also facilitate reliable and continuous monitoring. The wearable health monitoring device may be configured to store the photoplethysmogram data, a health status of the wearer, or both to the memory.

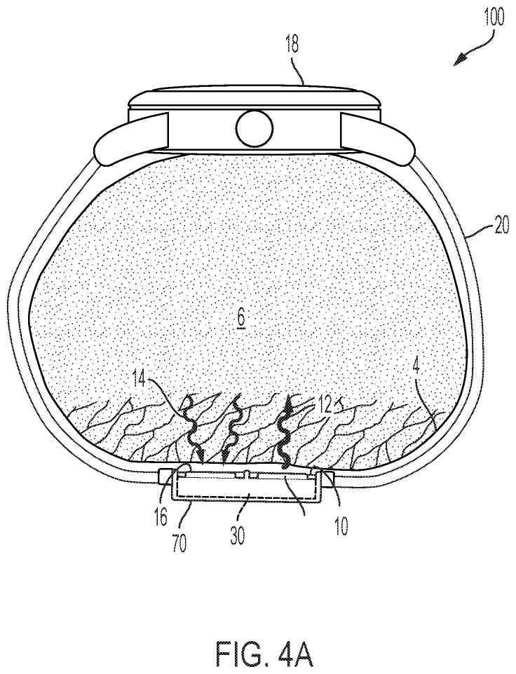

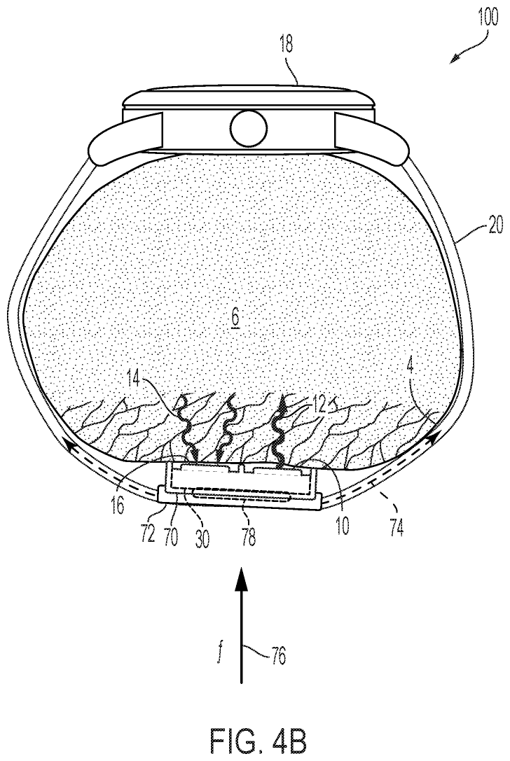

FIG. 4A is a side view of the wearable personal health monitoring device 100 of FIGS. 1 and 2, which may exhibit insufficient inward force resulting in a gap between the device and the wearer's skin and is improved upon according to an embodiment of the present disclosure. The wearable personal health monitoring device 100 is worn on the ventral side of the wrist and comprises the sensor unit 30 comprising the LED 10 and the photodiode 16. The sensor unit 30 is shown disposed within a housing 70. The housing 70 may be configured to receive and house the sensor unit 30. In the embodiment of FIG. 4, the housing 70 comprises a coupling mechanism to the housing 70 to a band 20 of a wristwatch 18. In another embodiment, the sensor unit 30 may be coupled to a band of a jewelry piece, a decorative band, etc. The housing 70 may be in line with the band 20 and may dispose the housing 70 against a portion of skin 4 of the wearer. Because the housing 70 is in line with the band, tension in the band 20 may not apply an inward radial force on the sensor unit 30. As discussed below, the embodiment illustrated in FIG. 4B is designed to position the sensor unit 30 such that tension in the band provides an inward radial force on the sensor unit 30. The housing 70 is configured to receive the sensor unit 30 and to dispose the sensor unit 30 toward the portion of skin 4 of the wearer. The disclosure describes use of a sensor unit 30 with the band 20 of the wristwatch 18 purely for convenience and not by way of limitation. In one embodiment, the band 20 may be coupled to the wristwatch 18 and configured to dispose both the wristwatch 18 and the housing 70 at the wrist or lower arm of the wearer. Moreover, the band 20 may be configured to dispose the housing 70 toward the ventral portion of the wrist or lower forearm and the wristwatch 18 to the top side of the wrist or lower forearm.

FIG. 4B illustrates another embodiment of the wearable personal health monitoring device 100 of FIGS. 1 and 2. In this embodiment, the housing 70 is disposed between the band 20 and the skin 4 of the wearer. For example, the band 20 may be configured to receive the housing 70 (e.g., accept a coupling mechanism of the housing 70 to engage the band 20), and to dispose the housing 70 against a portion of skin 4 of the wearer. The thickness of the housing 70 creates a distance between the skin 4 of the wearer and the band 20 which causes a tension 74 in the band 20. The tension 74 in the band 20 applied to the housing 70 is configured to exert an inward radial force 76 on the sensor unit 30 when coupled to the band 20 when it is at least partially disposed about the wrist or lower forearm of the wearer. In other words, the positioning of the housing 70 between the band 20 and the skin 4 of the wearer creates a substantially triangular shape or other shape that enables the housing 70 to exert the inward radial force 76. The housing 70 may have a predetermined thickness that creates a gap between the band 20 and the skin 4 of the wearer adjacent to the housing 70. In comparison to the embodiment illustrated in FIG. 4A, because the housing 70 is in line with the band 20, tension in the band 20 does not exert an inward radial force similar the embodiment of FIG. 4B. Accordingly, the inward radial force 76 created by the embodiment in FIG. 4B helps ensure that the sensor unit 30 is in good contact with the ventral side of the wrist or lower forearm so that the sensor unit 30 detects a quality photoplethysmographic signal.

With the sensor unit 30 coupled to the band 20, the LED 10 is disposed toward the skin 4 such that the LED 10 may radiate light 12 toward the skin 4 and at least partially into the subdermal matter 6. A portion of the radiated light 12 may become diffused/reflected light 14 due to the subdermal matter 6 and may be received at the photodiode 16. The band 20 may serve to dispose the sensor unit 30 toward the skin 4 and to retain the sensor unit 30 in a preferred position relative to the skin 4 and the subdermal matter 6. As discussed above, the band 20 may facilitate application of force 76 to properly position or otherwise dispose the sensor unit 30. If the amount of force 76 applied is too small, radiated light 12 may not be transmitted directly into the skin due to air gaps. Air gaps can also cause some radiated light 12 from the LED 10 or ambient light to be received at the photodiode 16. Additionally, if the force 76 is too small the sensor unit 30 may move in relation to the skin 4, which may introduce anomalous data (e.g., movement artifacts 68 such as described in FIG. 3C). Conversely, too much force 76 may decrease perfusion of the local tissue adjacent the sensor unit 30 and diminish the reliability of the resulting photoplethysmogram.

Relatedly, positioning the sensor unit 30 adjacent a ventral or palmar region of a wearer's wrist (or lower forearm) may improve the reliability of the photoplethysmography because the area is well perfused and several major arteries are nearby. However, placing the sensor unit 30 in this area comes with its own problems. At times, the anatomy of the region may adversely impact the quality of the photoplethysmographic signal since the area is flat or somewhat concave. This becomes more obvious when a wearer balls the hand or forms a first which may exacerbate the degree of concavity of the region. This concavity can cause the aforementioned gap resulting in a loss of signal. Certain embodiments of the present invention may overcome this anatomical effect.

This is illustrated in FIG. 4A, where a sensor unit 30 and a tensioning band are placed parallel on a relatively flat surface (e.g., on a plane). In this case, no matter what the tension in the band 20, there will be no inward radial force on the sensor unit 30 since the vertical component of the force vector is zero. However, if the sensor unit 30 is placed into a housing 70 and the bands 20 supplying the tension is placed on the housing 70 at a point away from the skin surface, there is now a vertical component of the tension force pushing the optical sensor into the tissue. This force can be adjusted by the tension in the band 20.

Referring again to FIG. 4B, the housing 70 is configured to dispose the sensor unit 30 between the skin 4 of the wearer and the band 20 (in this embodiment, the band 20 of the wristwatch 18). A band interface 72 may ensure the sensor unit 30 remains disposed between the band 20 and the skin 4 as illustrated by the principle in FIG. 4B. Furthermore, tension 74 in the band may provide force 76 adequate to ensure the sensor unit 30 remains properly disposed relative to the skin 4. The wearable personal health monitoring device 100 may further comprise a pressure sensor 78. The pressure sensor 78 may comprise a force-sensitive transducer capable of measuring an inward force (e.g., inward radial force) of the sensor unit 30 against the portion of skin 4 of the wearer. The pressure sensor 78 may be disposed so as to measure a degree of force 76 pressing the sensor unit 30 against the skin 4. The sensor unit 30, or the pressure sensor 78, may be configured to alert the wearer if the force 76 fails to achieve a minimum threshold (too little force, potentially permitting movement of the sensor unit 30) or exceeds a maximum pressure (too much force, potentially affecting perfusion of the region of skin 4 adjacent the sensor unit 30). The minimum and maximum thresholds may be customizable to the individual wearer to maximize reliability of photoplethysmography.

A processor (e.g., processor 49 in FIG. 2) may be configured to receive from the sensor unit 30 data about received optical energy in the form of diffused/reflected light 14. Moreover, the processor may be configured to produce photoplethysmogram data using the received data, and to store the received data to the memory or a data storage device.

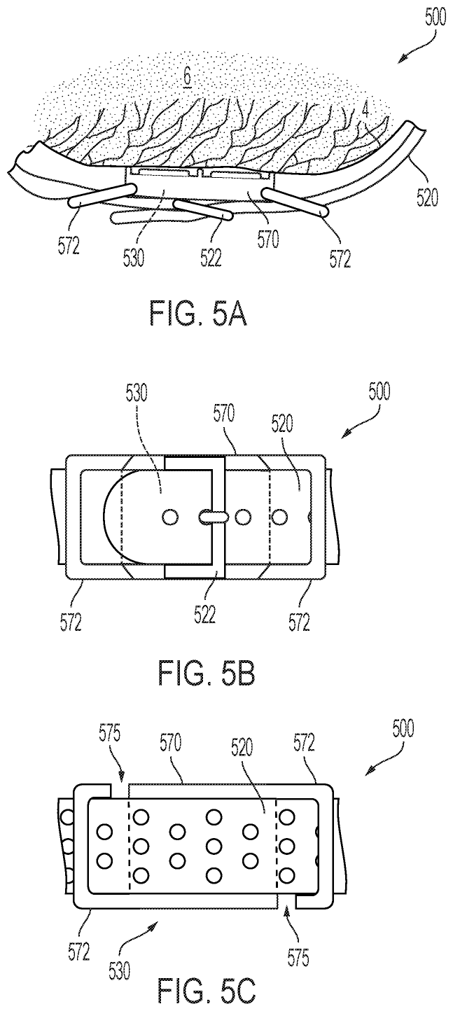

FIG. 5A depicts an embodiment of a wearable personal health monitoring device 500 that resembles the wearable personal health monitoring device 100 described above in certain respects. Accordingly, like features are designated with like reference numerals, with the leading digits incremented to “5.” For example, the embodiment depicted in FIG. 5A includes a sensor unit 530 that may, in some respects, resemble the sensor unit 30 of FIG. 4. Relevant disclosure set forth above regarding similarly identified features thus may not be repeated hereafter. Moreover, specific features of the wearable personal health monitoring device 100 and related components shown in FIG. 4 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the wearable personal health monitoring device 500 and related components depicted in FIG. 5A. Any suitable combination of the features, and variations of the same, described with respect to the wearable personal health monitoring device 100 and related components illustrated in FIGS. 1-4 can be employed with the wearable personal health monitoring device 500 and related components of FIG. 5A, and vice versa. This pattern of disclosure applies equally to further embodiments depicted in subsequent figures and described hereafter, wherein the leading digits may be further incremented.

FIG. 5A is a partial side view of a wearable health monitoring device 500 having loop connectors 572 to dispose and retain a housing 570 and a sensor unit 530 against the portion of the skin 4. The subdermal matter 6 is shown for reference. The loop connectors 572 are disposed at opposite ends of the housing 570. A band 520 may pass through each of the loop connectors 572 and pass over an outward portion of the housing 570. If the band 520 is equipped with a buckle or fastener 522, the buckle or fastener 522 may rest against the outward portion of the housing 570. The loop connectors 572 may permit a position of the housing 570 and sensor unit 530 to be selectively adjustable for comfort and to ensure the sensor unit 530 adequately presses against the portion of the skin 4.

FIG. 5B is a partial bottom view of the wearable health monitoring device 500 of FIG. 5A. The band 520 is shown disposed through each of the loop connectors 572. The buckle 522 of the band 520 is disposed against an outward portion of the housing 570. The sensor unit 530 is disposed within the housing 570.

FIG. 5C is a partial bottom view of the wearable health monitoring device 500 of FIG. 5A having loop connectors 572. The loop connectors 572 couple the housing 570 to the band 520. Each loop connector 572 comprises an arm that extends initially away from the housing 570, then laterally, and then back toward the housing 570. A gap or an opening 575 is disposed at one lateral edge of the housing and the loop connector 572, whereby the loop connector 572 may be slid or rotated to receive the band 520 and to dispose a portion of the band 520 over an outward portion of the housing 570 so that the housing 570 and sensor unit 530 are pressed against the portion of skin (see 4 in FIG. 5A). In the illustrated embodiment, the wearable health monitoring device 500 comprises two loop connectors 572, one at each longitudinal edge of the housing 570. The loop connectors 572 may permit a position of the housing 570 and sensor unit 530 to be selectively adjustable for comfort and to ensure the sensor unit 530 is pressed against the portion of the skin 4. The wearable health monitoring device 500 with loop connectors 572 may be particularly suited for use with a band 520 absent a buckle, such as the buckle 522 of FIGS. 5A and 5B.

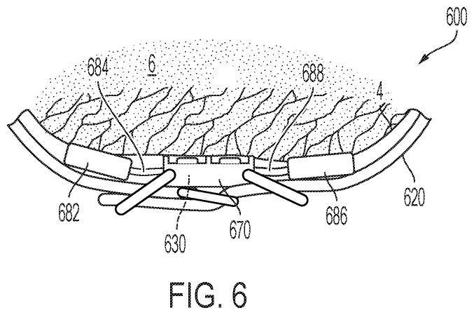

FIG. 6 is a partial side view of a wearable health monitoring device 600 according to an embodiment of the disclosure. The portion of the skin 4, the subdermal matter 6, and a band 620 are shown for reference. A sensor unit 630 is disposed within a housing 670 and oriented toward and pressed against the portion of the skin 4. A first extension unit 682 may be coupled to the housing 670 and disposed to one side of the housing 670 and situated between the band 620 and the portion of the skin 4. The first extension unit 682 may couple to the housing 670 by means of a first flexible coupling 684 to permit the extension unit 682 to generally conform to an anatomical feature of the wearer. A second extension unit 686 may be coupled by a second flexible coupling 688 to the housing 670 and disposed to an opposite side (relative to the first extension unit 682) of the housing 670.

The first and second extension units 682, 686 may each be configured to provide particular utility for the sensor unit 630. For example, both extension units 682, 686 may receive and carry a battery by which the sensor unit 630 may be powered. This would permit reducing the size of the sensor unit 630 itself by offloading the power supply to the extension units 682, 686. Furthermore, by placing the power supply at the extension units 682, 686, the sensor unit 630, if damaged, may be replaced by another sensor unit without the need to replace the power supply. Conversely, if the power supply ceases to function nominally, the power supply may be replaced—without replacing the sensor unit 630—by replacing one or both extension units 682, 686 or the entire housing 670. Furthermore, from time to time, a different sensor unit 630 providing different health monitoring functions may be exchanged as needed. In another embodiment, one or both extension units 682, 686 may be configured with a sensor in addition to the sensor unit 630, which may allow redundant data recording (recording the same data from a plurality of closely located positions over the portion of the skin 4) or different data recording (sensors having different functionality). In another embodiment, one or both extension units 682, 686 may contain memory whereby the size of the sensor unit 630 may be reduced in size, and/or the recording duration of the wearable health monitoring device 600 may be extended. Other uses, and other combinations of uses of the first and second extension units 682, 686, are anticipated by the disclosure.

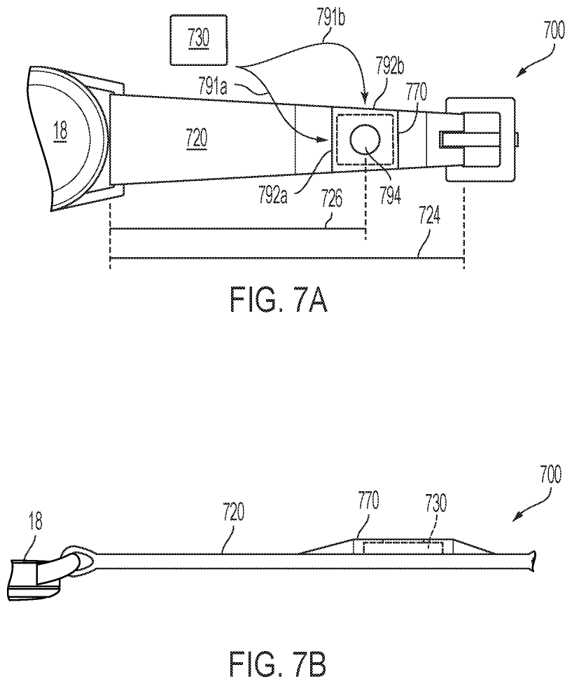

FIG. 7A is a partial inverse plan view of a wearable health monitoring device 700, according to an embodiment of the disclosure. In the instant embodiment, the wearable health monitoring device 700 employs a band 720 for the wristwatch 18 with a housing 770 for a sensor unit 730 incorporated into the band 720. The wristwatch 18 is here shown because of the frequency of watch wearing; however, other wrist or lower arm appurtenances are also contemplated. The housing 770 may include a lateral opening 792a or a longitudinal opening 792b in one side of the housing 770 whereby the sensor unit 730 may be inserted into the housing 770. The housing 770 may include a sensor aperture 794 through which a portion of the sensor unit 730 may couple to a portion of the skin (see skin 4 in FIGS. 5A, 6). The band 720 may have a fixed length 724 whereby the housing 770 and sensor unit 730 may be disposed at a selected position about the wrist or lower arm of the wearer. In other words, the sensor aperture 794 may be disposed a fixed distance 726 from the wristwatch 18 or another wrist appurtenance. To achieve a different fixed distance 726, a band 720 of a particular fixed length 724 may be employed.

FIG. 7B is a partial side view of the wearable health monitoring device 700 of FIG. 7A. The wristwatch 18 and sensor unit 730 are shown for reference. The housing 770 is configured to extend toward the skin of the wearer from an underside of the band 720 so that the sensor unit 730 may be disposed against the portion of the skin.

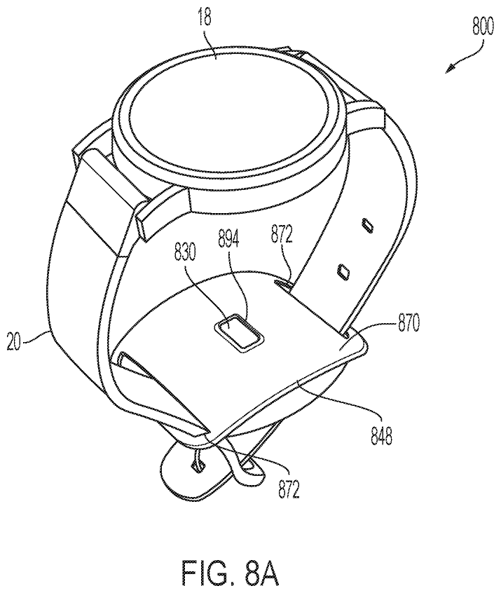

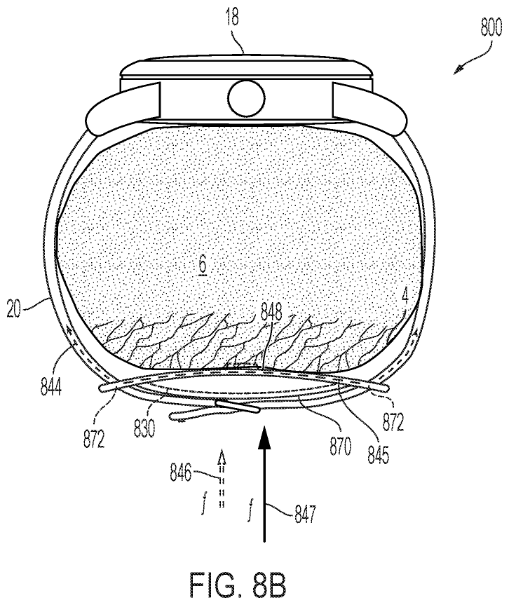

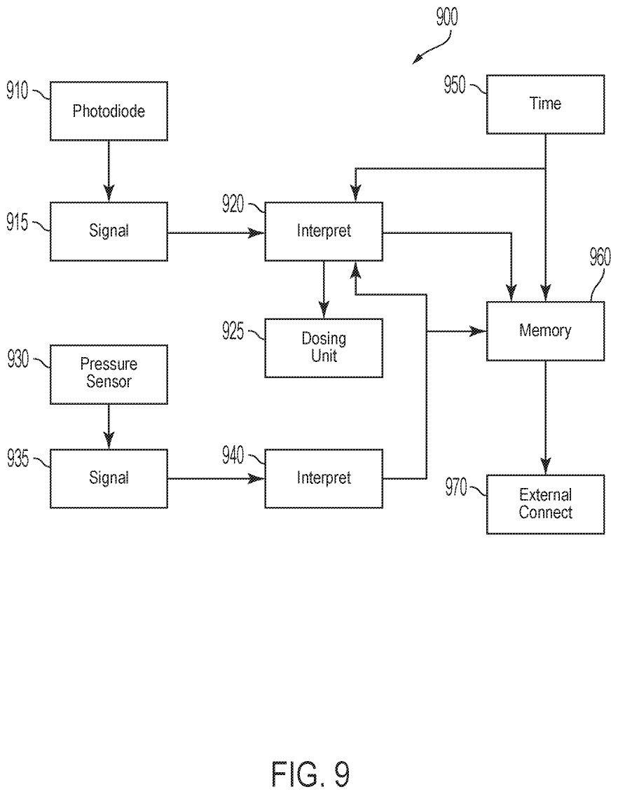

FIG. 8A is a perspective view of a wearable health monitoring device 800, according to an embodiment of the present disclosure, and with a housing 870 having an arcuate form 848. The wristwatch 18 is shown for reference. The housing 870 comprises two band openings 872 to receive the band 20. The two band openings 872 may be disposed at opposing longitudinal ends of the housing 870. The housing 870 further comprises a sensor aperture 894 whereby a sensor unit 830 may be exposed toward and pressed against a portion of the skin (see e.g., skin 4 in FIGS. 5A, 6). The arcuate form 848 of the housing 870 is convex and arches, with respect to the two band openings 872, toward the portion of the skin 4. The arcuate form 848 of the housing 870 may ensure the sensor unit 830 is appropriately pressed against the portion of the skin to procure consistent and accurate sensor data. As discussed in relation to FIG. 4B, because the band 20 supplying the tension is placed on the housing 70 at a point away from the skin surface, there is now a vertical component of the tension force pushing the optical sensor into the tissue due to the tension in the band 20. This force can be adjusted by the tension in the band 20.

FIG. 8B is a partial side view of the wearable health monitoring device 800 of FIG. 8A. The wristwatch 18 and subdermal matter 6 are shown for reference. The band 20 passes through the band openings 872 to retain the housing 870 between the band 20 and the portion of the skin 4. By being placed about the wrist, the band 20 may tend to draw 844 the housing 870 and sensor unit 830 toward the portion of skin 4 with some degree of force 846. The housing 870 may, by virtue of the arcuate form 848, load spring force 845 whereby the housing 870 may provide a compression resistive force that may combine with the nominal force 846 to produce substantial force 847 whereby the housing 870 and sensor unit 830 may be adequately pressed to the portion of the skin 4.

The embodiments of FIGS. 4-8 are examples and are not by way of limitation of the wearable health monitoring device. The disclosure anticipates use of the wearable health monitoring device with, for further examples, a variety of watches and watchbands, a variety of wristbands not associated to a watch, jewelry-style bands, etc. Furthermore, the disclosure anticipates a wearer of the wearable health monitoring device may, from time to time, change the watch, watchband, wristband, or jewelry band and will be able to personally fit and adjust the wearable health monitoring device with each of these to enable long-term monitoring. Additionally, the disclosure anticipates that the wearer may be able to adjust the fitment of the wearable health monitoring device without the need to procure additional equipment or to visit a doctor's office, etc.

FIG. 10 is a diagram of a method 900 of a wearable health monitoring device that, in at least some extent, may be used with or otherwise implemented by each of the embodiments disclosed herein. A photodiode 910 may detect diffused/reflected light and may produce a signal 915 qualitatively and/or quantitatively related to the detected diffused/reflected light. The signal 915 from the photodiode may be interpreted 920 by a processor (see 49 in FIG. 2). The interpretation 920 may transform the signal 915 to a datum capable of being stored at a memory 960. The interpretation 920 may identify a signal 915 as potentially diagnostic of a health or medical condition. The interpretation 920 may also identify a medical situation exceeding a programmed threshold and, if one is present, may cause a dosing unit 925 to dispense a dose of medicine. The interpretation 920 may be saved to the memory 960 for later review. When a dosing unit 925 is present, the signal to the dosing unit 925 may also be saved to the memory 960.

In an embodiment having a pressure sensor 930 (see 78 in FIG. 4), the pressure sensor 930 may detect force applied to the wearable health monitoring device and produce a signal 935 corresponding to the amount of force. The signal 935 may be interpreted 940 by the processor 49. The interpretation 940 may transform the signal 935 into a datum capable of being stored at the memory 960. The interpretation 940 may include identification of a probable movement artifact (see 68 in FIG. 3B) (suggesting the wearable health monitoring device is too loosely fitted), a condition of over-pressure (suggesting the wearable health monitoring device is too tightly fitted and may be impairing perfusion of tissue), etc. Identification of probable movement artifacts 68, et al., may indicate a need to adjust the fit of the wearable health monitoring device to ensure accuracy and reliability. The interpretation 940 of the signal 935 from the pressure sensor 930 may also be introduced into and affect the interpretation 920 of the signal 915 from the photodiode 910. The interpretation 940 may be saved to the memory 960. The wearable health monitoring device may be further configured to notify a wearer to adjust the fit for a reason other than looseness/tightness, such as, for example, to adjust the position of the sensor relative to a particular portion of the skin.

The method 900 of the wearable health monitoring device may further comprise time keeping 950. The time keeping 950 may be a function internal to the processor 49, and may be periodically regulated by connection with an external time source. The time keeping data may be stored to the memory 960, and may also be utilized at the interpretation 920 of the signal 915 from the photodiode 910.

The method 900 of the wearable health monitoring device may further comprise a capability to be connected to an external device. A connection 970 to the external device may be wired or wireless. For example, the wearable health monitoring device may be equipped with a port to permit physical connection to an external device, or may employ near-field communication (NFC), Bluetooth® connectivity, or other another appropriate wireless communication means to facilitate a non-physical connection to an external device. When a connection to the external device 970 is detected, data stored at the memory 960 may be uploaded to the external device 970. The external device 970 may also be used to program/reprogram a sensor unit. Furthermore, the connection 970 to the external device, and more particularly a wireless connection, may enable the wearable health monitoring device to communicate in near real-time with, for example, an application running on a portable device such as a “smartwatch,” a “smart phone,” etc. A wireless connection to a portable device may be particularly beneficial as a means of storing data from the wearable health monitoring device, communicating periodically or in near real-time with a health care provider service, and providing real-time alerts.

Real-time alerts, by way of example, may include causing a connected “smart phone” to provide an audible and visual signal to remind the wearer to take medicine, to alert the wearer to adjust the fit of the wearable health monitoring device, or to alert the wearer of detection of a health issue potentially requiring immediate intervention. Real-time alerts related to a health issue potentially requiring immediate intervention may include escalation modes. For example, the real-time alert may include an audible signal likely detectable by a wearer that, if unacknowledged for a prescribed period, is then amplified, and may be further amplified so as to potentially alert another individual in the vicinity. Escalation may include activation of a screen or a light of the associated “smart phone.” A real-time alert may be configured such that, under appropriate conditions, the “smart phone” may activate an emergency medical system (EMS), such as by dialing 911 or another prescribed phone number and delivering an automated message. The automated message may include details enabling EMS to identify the wearer of the wearable health monitoring device, provide an indication of the detected medical situation, provide global positioning system (GPS) data, etc. The wearable health monitoring device may also provide notification to other designated individuals, including in conjunction with an EMS activation. Similarly, the wearable health monitoring device may be configurable to permit automatic notification to one or more designated persons in non-emergency health situations.

Throughout this specification, the phrase “coupled to” refers to any form of interaction between two or more entities, including mechanical, electrical, magnetic, electromagnetic, fluid, and thermal interaction. Two components may be coupled to each other even though they are not in direct contact with each other.

The terms “a” and “an” can be described as one, but not limited to one. For example, although the disclosure may recite a tab having “a line of stitches,” the disclosure also contemplates that the tab can have two or more lines of stitches.

Unless otherwise stated, all ranges include both endpoints and all numbers between the endpoints.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

### EXAMPLES

### Example 1

A health monitoring device comprising a sensor unit comprising a source of optical energy and a receiver of optical energy; a housing configured to receive and house the sensor unit; a coupling mechanism configured to couple the housing to a band to be worn at least partially about one of a wrist and a lower forearm of a wearer of the band, the coupling mechanism further configured to position the housing pointing the sensor unit inward to the wrist or lower forearm of the wearer with an inward radial force on the sensor unit proportional to tension in the band; a data storage device; and a processor configured to receive from the sensor unit data about received optical energy, and to produce photoplethysmogram data using the received data, and to store data to the data storage device.

### Example 2

The health monitoring device of example 1, wherein the band is configured to receive the housing and to dispose the housing against a portion of skin of a wearer of the health monitoring device.

### Example 3

The health monitoring device of example 2, wherein the housing is configured to receive the sensor unit and to dispose the sensor unit toward the portion of skin of the wearer of the health monitoring device.

### Example 4

The health monitoring device of example 3, wherein the sensor is disposed toward a ventral portion of the one of a wrist or a lower forearm.

### Example 5

The health monitoring device of example 4, wherein the band and housing are configured to press the sensor unit against the portion of skin of the wearer of the health monitoring device.

### Example 6

The health monitoring device of example 5, further comprising a force-sensitive transducer capable of measuring an inward force of the sensor unit against the portion of skin of the wearer, and capable of signaling the processor to notify the wearer of a condition of force inadequate to permit the sensor unit to function nominally.

### Example 7

The health monitoring device of example 6, wherein the data storage device comprises machine-executable instructions.

### Example 8

The health monitoring device of example 7, wherein the processor is capable of receiving and executing the machine-executable instructions of the data storage device.

### Example 9

The health monitoring device of example 8, wherein the processor evaluates the photoplethysmogram data to assess a health status of the wearer of the health monitoring device.

### Example 10

The health monitoring device of example 9, wherein the processor stores on of the photoplethysmogram data or the health status at one of the data storage device or an external device.

### Example 11

The health monitoring device of example 10, wherein the external device is one of a “smartwatch” or a cellular telephone, and wherein the external device is capable of storing the data received from the sensor unit.

### Example 12

The health monitoring device of example 11, wherein the band is coupled to a wristwatch and is configured to dispose both the wristwatch and the housing at one of a wrist or a lower forearm.

### Example 13

The health monitoring device of example 12, wherein the band is coupled to a wristwatch and configured to dispose the housing toward the ventral portion of one of a wrist or a lower forearm.

### Example 14

The health monitoring device of example 13, wherein the band is coupled to a decorative device and configured to dispose the housing toward the portion of skin at the ventral portion of one of the wrist or the lower forearm.

### Example 15

A personal health device, comprising a sensor unit comprising a source of optical energy and a receiver of optical energy; a housing configured to receive and house the sensor unit; a coupling means configured to couple the housing to a band to be worn at least partially about one of a wrist or a lower forearm of a wearer of the band, the coupling means further configured to position the housing to point the sensor unit inward to one of a wrist or a lower forearm of the wearer with an inward radial force on the sensor proportional to tension in the band; an electronic storage device to store data produced by the personal health device; and a processor configured to receive data from the sensor unit and, using the data from the sensor unit, to produce photoplethysmogram data, and further configured to store data on the electronic storage device.

### Example 16

The personal health device of example 15, wherein the band is configured to receive the housing and to dispose the housing opposite a ventral portion of skin at one of a wrist or a lower forearm of the wearer of the personal health device.

### Example 17

The personal health device of example 16, wherein the sensor unit is disposed toward a ventral portion of the one of a wrist or a lower forearm.

### Example 18

The personal health device of example 17, wherein the band and housing are configured to press the sensor unit against the skin of the wearer of the personal health device.

### Example 19

The personal health device of example 18 further comprising a transducer capable of measuring the inward radial force of the sensor unit against the portion of skin of the wearer of the personal health device, and further capable of signaling the processor to notify the wearer in a condition wherein the inward radial force is inadequate to permit the sensor unit to perform nominally.

### Example 20

The personal health device of example, wherein the processor evaluates the photoplethysmogram data to assess a health status of the wearer of the personal health device.

### Example 21

The personal health device of example 20, wherein the processor stores one of the photoplethysmogram data or the health status to one of the electronic storage device or an external device.

### Example 22

The personal health device of example 21, wherein the external device is one of a “smartwatch” or a cellular telephone, and the one of a “smartwatch” or a cellular telephone comprises an electronic storage medium capable of storing the data sent by the processor.

### Example 23

The personal health device of example 22, wherein the band is coupled to one of a wristwatch or to band of a wristwatch or to a decorative device, and is configured to dispose the housing at one of a wrist and a lower forearm.

### Example 24

The personal health device of example 23, further comprising a medical-grade pump capable of storing a dose of medicine and delivering the dose of medicine to a wearer of the personal health device.

### Example 25

The personal health device of example 24, wherein the medical-grade pump is disposed at a site of a body of the wearer of the personal health device suitable for receiving the dose of medicine.

### Example 26

The personal health device of example 25, wherein the processor is capable of communicating with the medical-grade pump.

### Example 27

The personal health device of example 26, wherein the processor causes the medical-grade pump to deliver the dose of medicine to the wearer of the personal health device.

### Example 28

The personal health device of example 27, wherein the processor stores to one of the electronic storage device or the external device the delivery of the dose of medicine.

### Example 29

The personal health device of example 28, wherein the processor determines that no dose of medicine is currently contained within the medical-grade pump, and signals the wearer of the personal heath device accordingly.

Recitation in the claims of the term “first” with respect to a feature or element does not necessarily imply the existence of a second or additional such feature or element. Elements recited in means-plus-function format are intended to be construed in accordance with 35 U.S.C. § 112(f). It will be apparent to those having reasonable skill in the art that changes may be made to the details of the above-described embodiments without departing from the underlying principles of the invention. Embodiments of the invention in which an exclusive property or privilege is claimed are defined as follows.

1. A health monitoring device, comprising:
a sensor unit comprising a source of optical energy and a receiver of optical energy;
a housing configured to receive and house the sensor unit;
a coupling mechanism configured to couple the housing to a band to be worn at least partially about one of a wrist and a lower forearm of a wearer of the band, the coupling mechanism further configured to position the housing pointing the sensor unit inward to a ventral side of the wrist or lower forearm of the wearer with an inward radial force on the sensor unit proportional to tension in the band;
a data storage device; and
a processor configured to receive data from the sensor unit about received optical energy, and to produce photoplethysmogram data using the received data, and to store data to the data storage device.

2. The health monitoring device of claim 1, wherein the housing is configured to be disposed between the band and skin of the wearer, wherein a thickness of the housing creates a gap between the band and the skin of the wearer adjacent to the housing.

3. The health monitoring device of claim 1, wherein the coupling mechanism comprises two openings disposed at opposing longitudinal ends of the housing, the two openings configured to receive the band.

4. The health monitoring device of claim 3, wherein the housing comprises an arcuate shape.

5. The health monitoring device of claim 4, wherein the band and housing are configured to press the sensor unit against a portion of skin of the wearer of the health monitoring device.

6. The health monitoring device of claim 5, further comprising a force sensitive transducer capable of measuring an inward force of the sensor unit against the portion of skin of the wearer, and capable of signaling the processor to notify the wearer of a condition of force inadequate to permit the sensor unit to function nominally.

7. The health monitoring device of claim 6, wherein the data storage device comprises machine-executable instructions.

8. The health monitoring device of claim 7, wherein the processor is capable of receiving and executing the machine-executable instructions of the data storage device.

9. The health monitoring device of claim 8, wherein the processor evaluates the photoplethysmogram data to assess a health status of the wearer of the health monitoring device.

10. The health monitoring device of claim 9, wherein the processor stores one of the photoplethysmogram data or the health status at one of the data storage device or an external device.

11. The health monitoring device of claim 10, wherein the external device is one of a “smartwatch” or a cellular telephone, and wherein the external device is capable of storing the data received from the sensor unit.

12. The health monitoring device of claim 11, wherein the band is coupled to a wristwatch and is configured to dispose both the wristwatch and the housing at one of a wrist and a lower forearm, wherein the wristwatch is disposed on the top side of the wrist.

13. A personal health device, comprising:
a sensor unit comprising a source of optical energy and a receiver of optical energy;
a housing configured to receive and house the sensor unit;
a coupling means configured to couple the housing to a band to be worn at least partially about one of a wrist or a lower forearm of a wearer of the band, the coupling means further configured to position the housing to point the sensor unit inward to a ventral side of the wrist or a lower forearm of a wearer with an inward radial force on the sensor proportional to tension in the band;
an electronic storage device to store data produced by the personal health device; and
a processor configured to receive data from the sensor unit and, using the data from the sensor unit, to produce photoplethysmogram data, and further configured to store data on the electronic storage device.

14. The personal health device of claim 13, wherein the housing is configured to receive the band to couple to an inward side of the band so as to be disposed between the band and skin of the wearer, wherein the housing has a predetermined thickness that creates a gap between the band and the skin of the wearer adjacent to the housing.

15. The health monitoring device of claim 1, wherein the coupling mechanism comprises two openings disposed at opposing longitudinal ends of the housing configured to receive the band, and wherein the housing comprises an arcuate shape.

16. The personal health device of claim 13, wherein the band and housing are configured to press the sensor unit against skin of the wearer of the personal health device.

17. The personal health device of claim 16 further comprising a transducer capable of measuring the inward radial force of the sensor unit against the portion of skin of the wearer of the personal health device, and further capable of signaling the processor to notify the wearer in a condition wherein the inward radial force is inadequate to permit the sensor unit to perform nominally.

18. The personal health device of claim 17, wherein the processor evaluates the photoplethysmogram data to assess a health status of the wearer of the personal health device.

19. The personal health device of claim 18, wherein the processor stores one of the photoplethysmogram data or the health status to one of the electronic storage device or an external device.

20. The personal health device of claim 19, wherein the external device is one of a “smartwatch” or a cellular telephone, and the one of the “smartwatch” or the cellular telephone comprises an electronic storage medium capable of storing the data sent by the processor.
